(19) United States
(12) Patent Application Publication
(10) Pub. No.: US 2013/0225980 A1
(43) Pub. Date: Aug. 29, 2013

(54) Shoulder Local Coil System for a Magnetic Resonance Tomography System

(71) Applicants: Stephan Biber, Erlangen (DE); Rainer Kurth, Erlangen (DE); Stephan Zink, Erlangen (DE)

(72) Inventors: Stephan Biber, Erlangen (DE); Rainer Kurth, Erlangen (DE); Stephan Zink, Erlangen (DE)

(21) Appl. No.: 13/781,214

(22) Filed: Feb. 28, 2013

(30) Foreign Application Priority Data
Feb. 29, 2012 (DE) DE 102012203083.2

(51) Int. Cl. A61B 5/055

(52) U.S. Cl. CPC A61B 5/055
USPC 600/422

(57) ABSTRACT

A shoulder local coil system for a magnetic resonance tomography system for generating magnetic resonance images of a shoulder region of a person to be examined is provided. The shoulder local coil system includes a base plate, a fixed or removable cover plate over the base plate, and a flexible local coil arranged wholly or partially movably between the base plate and the cover plate.

This application claims the benefit of DE 10 2012 203 083.2, filed on Feb. 29, 2012, which is hereby incorporated by reference.

### BACKGROUND

The present embodiments relate to a shoulder local coil system for a magnetic resonance tomography system.

In a magnetic resonance device, the body to be examined (e.g., the patient or a part of the patient) may be exposed to a defined basic magnetic field with the aid of a basic magnetic field system (e.g., the B₀ field). In addition, a magnetic field gradient is applied with the aid of a gradient system. The high-frequency magnetic resonance excitation signals (HF signals) with defined field strengths are then transmitted by suitable antenna via a high-frequency transmission system. The magnetic flux density may be designated B₁. Therefore, the pulse-shaped high-frequency field may also be known in short as the B₁ field. These pulse-shaped HF signals are used to tip the nuclear spins of certain atoms that have been excited in a resonant manner by this high-frequency field through a defined flip angle in relation to the magnetic field lines of the basic magnetic field (B₀ field). During the relaxation of the nuclear spins, high-frequency signals (e.g., magnetic resonance signals) are again emitted, received by suitable receive antennas and then further processed. The magnetic resonance signals or “raw data” acquired in this way may be used to reconstruct the desired magnetic resonance image data (MRI image data). Local encoding is performed by switching suitable magnetic field gradients in the differential spatial directions at precisely defined times (e.g., during the emission of the HF signals and/or on the reception of the magnetic resonance signals). The emission of the high-frequency signals for nuclear spin magnetization may be performed by a whole body coil or body coil built into the magnetic resonance tomography scanner.

Local coils (e.g., “coils” or “local coils”) are used to receive the magnetic resonance signals. These are antenna systems that are dedicated to specific body regions (e.g., the region of the extremities or specific body regions such as the head, heart, prostate, knee, ankle or shoulder joint). The magnetic resonance signals induce a voltage in the individual antennas of the local coil. The induced voltage is then amplified with a low-noise preamplifier (LNA, preamp) and forwarded via a cable connection to the receive electronics. High-field systems are used to improve the signal-to-noise ratio (e.g., the SNR) for, for example, with high-resolution images. A basic magnetic field B₀ of 1.5 to 12 tesla and more is used.

The signal-to-noise ratio of an image is of significance for numerous magnetic resonance applications (e.g., clinical MRIs). This is decisively determined by the local coil (e.g., with antenna and with active amplifiers) by the losses in the actual antenna elements. Very small antennas enable a very high SNR close to the field of view but are restricted to very small volumes. To increase the FoV and due to the possibility of accelerated measurement by parallel imaging (e.g., with k-space undersampling and subsequent signal processing with formalisms such as sensitive encoding (SENSE)) and generalized autocalibrating partially parallel acquisitions (GRAPPA)), there is interest in high-channel, very dense antenna arrays (e.g., antenna systems with numerous channels), the individual elements of which may have a completely different alignment relative to the transmitting field (e.g., the B₁ field).

Shoulder local coils with a plurality of individual elements exist in the form of fixed shell elements, in which the patient to be examined is cumbersomely moved in the z-direction (e.g., along the B₀ field or the actual body axis) and x-direction (e.g., perpendicular to the z-direction in the plane of the patient support). If patients to be examined are elderly or corpulent or in the case of patients with shoulder injuries, this is often awkward, painful and time-intensive. FIG. 1 shows a schematic diagram of a conventional local coil for the shoulder region as an example of coils of this kind with shell-shaped antenna elements. This local coil includes a base plate 700 with antenna elements and a shell-shaped coil element 710 with antenna elements arranged perpendicularly thereupon. The coil element 710 comes in two different shell sizes in order to adapt the local coil to different patient sizes.

Shoulder local coils with an overall structure adaptable to the patient anatomy in y-direction are described, for example, in the patent application US 2010/0280360 A1. The antenna elements are mainly arranged below (e.g., posterior to) and above (e.g., anterior to) the shoulder joint region (e.g., substantially outside the plane of the patient lying on a patient support (in the y-direction)). The local coil includes two halves that may be divided in the y-direction and displaced into a plurality of latching positions in order to enable the size of the local coil to be adapted to the size of the person to be examined in the y-direction. However, this height adjustment (variability in the y-direction) of the two shell parts results in a gap in the antenna coverage (e.g., in the region of the side (lateral) coverage of the shoulder region of the person to be examined). The gap is detrimental with respect to the SNR.

### SUMMARY AND DESCRIPTION

The present embodiments may obviate one or more of the drawbacks or limitations in the related art. For example, an improved alternative to existing local coil systems for the shoulder region and a high-frequency receiving device, a magnetic resonance system and a method for generating improved magnetic resonance signals with a shoulder local coil system are provided.

The shoulder local coil system for a magnetic resonance tomography system for generating magnetic resonance images of a shoulder region of an object to be examined includes a base plate, a fixed or removable cover plate over the base plate and a flexible local coil arranged wholly or partially movably between the base plate and the cover plate.

The shoulder region may, for example, be the whole or a part of the shoulder of a patient. The shoulder joint may be the subject of the examination. However, the shoulder joint is located relatively deeply in the body and is also not accessible on all sides. The wide variation in the patient anatomy in the shoulder region is a further drawback. The patient anatomy in the shoulder region also lies in an asymmetric position relative to the longitudinal axis of the body. A further drawback in comparison to local coils for extremities or other body parts includes the fact that, for anatomical reasons, the local coil is unable to enclose the joint completely. With existing shoulder local coils, all this results in lower signal-to-noise ratios (SNR). With the shoulder local coil system, due to the flexibility and the movable arrangement of the local coil in the region between the base plate and the cover plate, the flexible local coil with the antennas integrated therein may be positioned simply and closely on or around the shoulder region to be examined. Despite the asymmetric location of the shoulder joint, these two properties result in an improved SNR during the examination.

The base plate is a part of a patient bed, another coil, or a removable or a permanently installed support disposed thereupon. This serves as a base element for supporting and guiding the local coil. In addition, the fastening elements for the local coil or corresponding moving mechanisms (e.g., guide tracks, return springs) or tilting mechanisms (e.g., rotary axes and bearings for the local coils on the rotary axis) for the local coil may be integrated. In addition, the base plate may be configured to accept or divert the corresponding cable routings and cables for the transmission of the signals received by the antennas to the high-frequency receiving device. In the region of the guide for the local coil, the base plate may include a recess or opening in order correspondingly to accept the local coil without enlarging the overall height of the whole construction.

The cover plate on which the person to be examined or the patient and, for example, the shoulder region (e.g., the region between the thorax and the head) of the patient lies during the examination is arranged over the base plate. The cover plate may be permanently connected to the base plate at individual sections or regions. Alternatively, the cover plate may also be embodied as removable enabling free access to the local coil arranged therebelow. The base plate and the cover plate form a type of tunnel or shaft, in which the local coil is arranged wholly or partially movably.

In one embodiment, the local coil may also be fixed to the cover plate by a mount, optionally by return springs, enabling the local coil to be pulled out of the shaft. The advantage of this embodiment is that, when the cover plate is rotated, the local coil may be rotated from the left side of the base plate or in the opposite direction. This enables a simple change of the local coil from one shoulder side to the other. The rotation may be substantially by 180° in order to achieve the change of side. However, stepless rotation by 360° may also be provided in order, for example, to be able to place a small-area local coil (with a small field of view (FoV)) more precisely under the region to be examined on the shoulder of the patient.

Instead of a rotatable cover plate, manual raising and repositioning to the desired place to change the shoulder side may also be provided. This enables a change of the local coil from one shoulder side to the other together with the cover plate to be performed quickly. Optionally, permanent fixing points are provided for positioning the cover plate on the base plate. These may, for example, be guides or mounts on the base plate in which bolt-like counterparts may be inserted or fixed on the cover plate.

The local coil may be arranged displaceably in the tunnel or shaft between the base plate under the cover plate (e.g., laterally displaceable, partially or completely withdrawable or moveable in some other way such as tiltable or rotatable). This enables the position of the local coil under the patient (e.g., including when the patient is lying on the cover plate for the examination) to be changed relative to the patient. This enables the staff operating the device, for example, to move the local coil within the tunnel or shaft from the base-plate side of the left shoulder toward the base-plate side of the right shoulder and without having to move the patient. Both the left shoulder and the right shoulder may be examined with the same local coil without changing the position of the patient. For example, this special mechanical embodiment of the shoulder local coil system permits an integrated workflow. This integrated workflow minimizes the amount of time required to prepare the patient and avoids the patient having to slide into the coil.

A flexible local coil may be a coil including a number of antenna elements or individual antennas arranged in a substantially level arrangement laterally in series and/or next to each other or in a matrix arrangement in a substantially rectangular alignment. This may be partially or completely embedded in a flexible material (e.g., a flexible plastic). Alternative possibilities for the production of flexible antenna arrangements as known to the person skilled in the art may also be used to develop the flexible local coil.

The external design of the basic shape of the shoulder local coil may be rectangular. The basic shape of the local coil may be less flexible in the direction of the length and breadth than in the thickness direction so that the local coil may be placed around the shoulder. In one embodiment, the displaceability or the tiltability in the shaft or tunnel for the flexibility may be achieved in special sections of the local coil (e.g., by an inbuilt joint or a bending region), and the regions lying substantially under the cover plate or on the patient may be embodied as rigid plate-shaped sections.

The local coil with a substantially rectangular basic shape includes a number of antennas (e.g., one or more; about 1 to 64; 8, 16 or 32). These may be substantially circular or substantially rectangular antennas or antennas with a complicated geometry (e.g., butterfly antennas) that may optionally also be present in combination in a local coil.

The individual antennas are connected together or each of the individual antennas is connected individually (e.g., with the aid of a cable guided out of the local coil) to evaluation electronics of a high-frequency receiving device.

The improvement of the mechanical mobility of the shoulder local coil system makes the system suitable for a high-frequency receiving device of a magnetic resonance tomography system for generating magnetic resonance images of a shoulder region. A high-frequency receiving device for a magnetic resonance tomography system for generating magnetic resonance images of the shoulder region includes at least one such shoulder local coil system that is connected to a receiving unit of the high-frequency receiving device (e.g., a usual receive channel for connecting a local coil) that further processes the signal acquired with the local coil and, if the signal is not yet in digital form, digitizes the signal, for example.

A magnetic resonance system for generating magnetic resonance images of the shoulder region includes, for example, a whole body scanner or dedicated scanner with a basic field magnet. In addition to the usual components known to the person skilled in the art (e.g., a basic field magnet system, a gradient system and a high-frequency transmission system), the magnetic resonance system uses a high-frequency receiving device with a receive coil like the shoulder local coil described.

In the method for generating magnetic resonance signals of a shoulder region, the use of a shoulder local coil system enables the time needed to prepare the patient and the SNR to be improved. The improvement of both properties simultaneously achieves an improvement in efficiency since an increase in the number of patients to be examined is accompanied by a simultaneous improvement in the image quality.

Advantageous embodiments and developments of the shoulder local coil elements, the high-frequency receiving device, the magnetic resonance system, and the method for generating magnetic resonance signals may be derived from the following description. The shoulder local coil element, the high-frequency receiving device, the magnetic resonance tomography system, and the method may also be embodied according to the respective other categories.

In one embodiment, the shoulder local coil system may include a local coil with a first coil section arranged during operation wholly or partially in alignment with the cover plate and a second coil section arranged during operation outside the region between the base plate and the cover plate that is movable relative to the first coil section.

Viewed from the patient, the first coil section may be arranged posterior to (e.g., below) the shoulder of the patient who lies on the cover plate in supine position (e.g., embodied such that, starting from the rear of the patient, the first coil section scans the shoulder for raw data acquisition). This first coil section includes a number of first antennas that, during operation according to the intended use, scan the shoulder region or the shoulder joint from the rear side of the shoulder.

The second coil section is provided as movable relative to the first coil section and may, by tilting or moving the second coil section around the shoulder of the patient, scan the anterior shoulder region or the upper shoulder section (e.g., in medical terms, the anterior). This second coil section includes a number of second antennas. In a simple way, a plurality of antennas may be arranged both anterior and posterior to the shoulder joint. This results, for example, in an improvement of the SNR since, in a plurality coil sections arranged movably relative to each other, the local coil may be arranged in the immediate vicinity of the shoulder joint. It is advantageous that, as the first coil section is embodied displaceably under the patient, the first coil section may be positioned more exactly under the shoulder joint than conventional fixed coil elements. Simultaneously, the second coil section, which is movable relative to the first coil section, may be placed in an anterior shoulder section on the patient.

In one embodiment, first and second coil sections may be coil sections actually adopting the aforementioned position. It may happen that due to the displacement or tilting of the whole local coil in order to change from the left shoulder side to the right shoulder side, the first coil section is pulled out under the cover plate and, due to the displacement or tilting, reaches a position above the shoulder (described above as the second coil section). Hence, on the change of shoulder, the first coil section and the second coil section may change respective designations as first to second or as second to first coil section. In the following, use on one side is predominantly explained as an example for both shoulder sides. The person skilled in the art will have no difficultly transferring this understanding to the other patient side.

In a further embodiment of the shoulder local coil system, the second coil section may be tilted out of a first position (e.g., a preparatory position, in which the first coil section and the second coil section lie in a first plane) that is substantially parallel to the base plate into a second position (e.g., an operational position) in which the second coil section wholly or partially lies in a second plane above the shoulder that is substantially parallel to the first plane. “Tiltable” may be that the positioning of inherently flexible coil sections about the shoulder. In one embodiment, the positioning is performed in a substantially U-shaped manner so that the posterior, lateral and anterior regions of the shoulder are covered by the local coil.

Therefore, for example, the second coil section does not disrupt the preparation of the patient on the patient support because the second coil section lies in a tilted-back condition within the first plane, a plane substantially lying in the z-direction at the height of the tunnel under the patient support. Optionally, the second coil section may be accommodated completely or partially in the tunnel. In this way, no disruptive local coil elements obstruct the user during the alignment of the patient on the patient support. Following the alignment of the patient on the patient support, after the alignment of the first coil section below (e.g., posterior to) the shoulder, the second coil section may be aligned in position above (e.g., anterior to) the patient's shoulder. This enables simpler operation of the shoulder local coil system and improved adaptation to the patient geometry than is the case with conventional local coils.

In one embodiment, in the first position, the shoulder local coil system is in an extended arrangement with respect to the first coil section and the second coil section. In the second position, the coil sections are in a tilted arrangement, in a substantially U-shaped embodiment (e.g., arranged opposite each other in two parallel planes). The shoulder is thus scanned from two planes with a plurality of antenna elements. The SNR is improved.

In order to achieve the desired flexibility for the adaptation of the arrangement of the second coil section relative to the first coil section, as described above, in a further embodiment, the shoulder local coil system may includes a flexible third coil section with a number of third antennas. The flexible third coil section is arranged between the first coil section and the second coil section. This not only achieves sufficient flexibility of the local coil system, it also enables the shoulder region to the side of the shoulder (e.g., a lateral region) to be covered with antenna elements. The individual local coil sections may be present separately or integrated in a flexible local coil system. Not only the third coil section, but also the first coil section and the second coil section may be embodied as flexible in order to improve the adaptation to the patient anatomy compared to the conventional rigid shell system. However, flexibility is, for example, provided in the third coil section of the coil system, since the curvature of the shoulder region is most strongly pronounced.

In a further embodiment of the shoulder local coil system, the flexible third coil section includes a center marking for the central alignment of the third coil section on the shoulder in the direction perpendicular to the base-plate plane (y-direction). The center marking is an advantageous identifying feature for the user when positioning the local coil system on the patient, since the center marking enables the symmetrical alignment of the local coil system to be recognized. When the local coil system is positioned symmetrically (e.g., when the center marking is aligned at half the shoulder height in the y-direction) in operational status, this causes the first coil section and the second coil section to adopt the desired opposite arrangement in the shoulder region. In other words, the antennas in the second coil element are approximately aligned with the antennas in the first coil element.

As an alternative to an inherently flexibly embodied local coil system (e.g., due to the choice of material), in a further embodiment of the shoulder local coil system, the first coil section and/or the second coil section and/or the third coil section may each have a basic structure with a number of parallel adjacent, linking elements (e.g., one or more; 2, 3, 4 or 5) or segments connected movably to each other. The inherently rigid linking elements or segments are connected flexibly to each other in a type of roller blind arrangement. In each of the linking elements or segments, a number of antennas (e.g., one or more; 2 to 8 or 2 or 4) may be installed in series (e.g., in the z-direction) and/or next to each other (e.g., in the x-direction).

The individual linking elements may be connected in a shear-resistant way in order to enable improved insertion of the shoulder local coil system into the tunnel or shaft under the cover plate. A roller-blind-like local coil of this kind enables simple movement into the shaft or tunnel (e.g., without jamming or tilting). It is also of advantage that the individual linking elements may be introduced into the shaft without time-consuming bending back of the local coil sections in a virtually level form. This may optionally also take place automatically using a return spring element installed in the shaft.

In one embodiment, the shoulder local coil system may include a cover having a cutout corresponding to the region of the first antennas or a corresponding opening. The shape and size of the cutout are such that optimum acquisition of the signals by the underlying antennas is facilitated, and simultaneously, the patient may be positioned comfortably thereon. The decisive factor for the local coil system according to the present embodiment is that, despite the patient being positioned over the cutout of the cover plate, there are no impediments due to objects such as parts of clothing or the patient's hair penetrating the opening, and the local coil arranged therebelow remains mobile.

According to a further embodiment of the shoulder local coil system, the shoulder local coil in the tunnel or shaft between the base plate and the cover plate may be embodied as partially or completely displaceable from a first side of the base plate to an opposite second side of the base plate. The first side, for example, forms the side of the base plate on which the left shoulder of the patient is placed. The second side forms the opposite side of the base plate (e.g., the side on which the right shoulder of the patient is placed). In other words, the shoulder local coil is arranged in the transverse direction to the patient's longitudinal axis (e.g., from one shoulder side to the other shoulder side displaceably under the cover plate).

In one embodiment, the shoulder local coil is embodied in the form of a sliding system such that the shoulder local coil may be pushed or pulled through under the patient (e.g., under the cover plate). This enables a simple change of the field of view (FoV) between the left and the right shoulder and without changing the position of the patient in the meantime. The flexible embodiment of the shoulder local coil enables the shoulder coil to be positioned by suitable reshaping (e.g., bending or tilting) on both shoulder sides. This facilitates an integrated workflow, which reduces the time for the preparation of the patient in comparison to conventional systems. This simultaneously improves the patient's comfort because there is no need to change the patient's position or for cumbersome sliding of the patient into a local coil shell.

As an alternative to the above embodiment of the shoulder local coil system, the shoulder local coil on the base plate may be embodied as tiltable from a first side of the base plate to an opposite second side of the base plate. For this embodiment in the form of a tilting system, the cover plate is removable or tiltable in order, after the removal of the cover plate, to tilt the actual local coil located thereunder from one side of the base plate to the other side of the base plate. Tilting again takes place from, for example, the left side of the base plate, when viewed in the direction of the longitudinal axis of the patient, to the right side of the base plate, or vice versa. This is followed by replacement of the cover or insertion of the cover plate in the mounts provided. The local coil system according to this embodiment is thus also embodied for use on both the left side and the right side of the patient side. The tilting mechanism enables a rapid change between both sides so that once again an integrated workflow with little time loss is possible.

To improve the coverage of the shoulder region or of the shoulder joint with antenna elements, according to a further embodiment, the shoulder local coil system may include a fourth coil section (e.g., a cranial coil section) with a number of fourth antennas. The fourth coil section is provided in an alignment that is substantially perpendicular to the first to third coil sections alignment (e.g., in “the positive z-direction” with respect to the shoulder) and, for example, in a region of the patient aligned toward the head. This fourth coil section with the fourth antenna elements facilitates an additional receiving plane (x-y-plane) for the MRI measurement so that the SNR may be further improved. This also applies even though the antenna elements, which are aligned in the x-y-plane, pick up only little B₁ field.

In an alternative embodiment, the fourth coil section may include an optional coil element permanently connected to the base plate. The advantage of this is that as few parts as possible of the local coil are embodied as moveable and this element may simultaneously be used for the alignment of the patient on the patient bed and forms the end of the shoulder for the patient.

According to another alternative embodiment, the fourth coil section may include a flexible coil element that may be arranged substantially perpendicular to the second coil section, which is embodied as a coil element that may be tilted around the cranial region of the shoulder. In this embodiment, therefore, the fourth coil section is embodied in one piece with the first coil section. The fourth coil section is embodied as movable toward the first coil section (e.g., tiltable or as inherently flexible due to the material). The flexibility increases the patient's level of comfort when changing position, and improved adaptability of the fourth coil section to the patient's shoulder anatomy of the patient is provided. This again improves the workflow and the SNR during the MRI scan.

The L-shape of the shoulder local coil according to the preceding embodiment created by the fourth coil section still allows the local coil to be displaced from the left shoulder toward the right shoulder, either by transverse displacement or by tilting the L-shaped shoulder local coil downward.

If the local coil system is transversely displaced, the fourth coil section is positioned on the patient anatomy on one side (e.g., in the case of the left shoulder) from below (posterior) to above (anterior) and on the other side (e.g., the right shoulder) from above (anterior) to below (posterior). The fourth coil section may be embodied such that the antennas are unshielded on a top side of the antennas and a bottom side of the antennas and thus may receive signals on both sides.

For the fastening of the fourth coil section, the fourth coil section may include a fastening element, for example, based on a Velcro fastener or a pushbutton system that may be tilted over the second coil section and fixed to this with the Velcro fastener or the pushbuttons. In order to provide the greatest possible variability for adaptation to different patient anatomies, a part of the antenna elements in the fourth coil section may lie over the second coil section (e.g., in the case of small shoulder sizes). In order to avoid interaction (e.g., electromagnet coupling) with the antenna elements provided in the second coil section, the antenna elements in the second and fourth coil section may be embodied so the antenna elements may be switched off individually in the case of overlapping.

If the local coil system on the base plate is tilted from the left side to the right side, or vice versa, the position of the fourth coil section is either always internal or always external so that the fourth coil section is tilted regardless of the shoulder side either from below (posterior) to above (anterior) or from above (anterior) to below (posterior).

The advantage of both embodiments is that a further signal acquisition plane is created without impairing the displaceability in the tunnel or in the channel, and simultaneously, an improvement of the SNR is achieved.

As explained above, the shoulder local coil systems according to the preceding embodiments enable adaptation to both shoulders (e.g., to the left and the right shoulder). The antennas may be simultaneously positioned exactly under the patient. These may also be simply adapted to the specific patient anatomy, regardless of whether the patient is small or large. The shoulder local coil systems may have an antenna structure with flexibility made of individual coil sections with antenna elements disposed in a shaft under the patient, but on the patient bed or on another coil (e.g., a spine coil). The displaceable or tiltable arrangement of the local coil system enables a simple change from one shoulder side to the other shoulder side, while simultaneously facilitating close physical proximity to the shoulder and hence an improved SNR. The favorable arrangement of one or a plurality of coil sections with different antenna systems together with a workflow-optimized mechanical design of the local coil, therefore, simultaneously optimizes the SNR and workflow.

### BRIEF DESCRIPTION OF THE DRAWINGS

The same components in the different figures are given the same reference number.

FIG. 1 shows a conventional shoulder local coil;

FIG. 2 is a schematic view of one embodiment of a magnetic resonance tomography system;

FIG. 3 is a schematic top view of one embodiment of an arrangement of a shoulder local coil system in preparatory position on a patient;

FIG. 4 is a schematic top view of one embodiment of an arrangement of a shoulder local coil system in preparatory position on a patient;

FIG. 5 is a schematic top view of one embodiment of an arrangement of a shoulder local coil system in operational position on a patient;

FIG. 6 is a head-side front view of one embodiment of an arrangement of a shoulder local coil system in operational position on the left shoulder of a patient;

FIG. 7 is a head-side front view of one embodiment of an arrangement of a shoulder local coil system in operational position on the left shoulder of a patient;

FIG. 8 is an outline front view of one embodiment of an arrangement of a shoulder local coil system in both operational position and in tilted position;

FIG. 9 is an outline front view of one embodiment of an arrangement of the shoulder local coil system in FIG. 8 in operational position on the left shoulder of a patient;

FIG. 10 is an outline front view of one embodiment of an arrangement of the shoulder local coil system in FIG. 8 in operational position on the right shoulder of a patient;

FIG. 11 is an outline front view (left) or side view (right) of one embodiment of an arrangement of the shoulder local coil system in FIG. 8 on tilting from the left to the right shoulder position; and

FIG. 12 is a schematic top view of one embodiment of an arrangement of a shoulder local coil system in operational position on a patient.

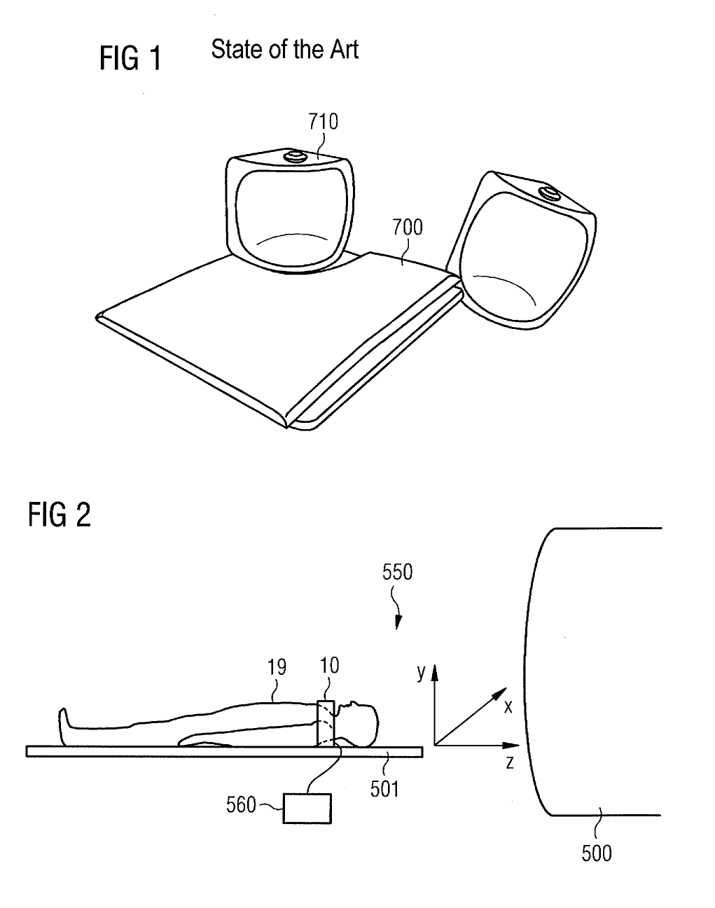

### DETAILED DESCRIPTION OF THE DRAWINGS

FIG. 2 shows an exemplary embodiment of a magnetic resonance tomography system 550 for generating magnetic resonance images of a shoulder region of a patient 19. The magnetic resonance tomography system 550 includes a scanner with a basic field magnet 500, in which the patient 19 lying on a patient bed 501 is pushed in the z-direction for examination. FIG. 2 illustrates the coordinate system with the directions x, y, z, as used in this application.

The magnetic resonance tomography system 550 also includes a high-frequency receiving device 560 for generating magnetic resonance images of a protected area of the patient 19, in which raw data recorded by a local coil system 10 are processed to produce MRI scans.

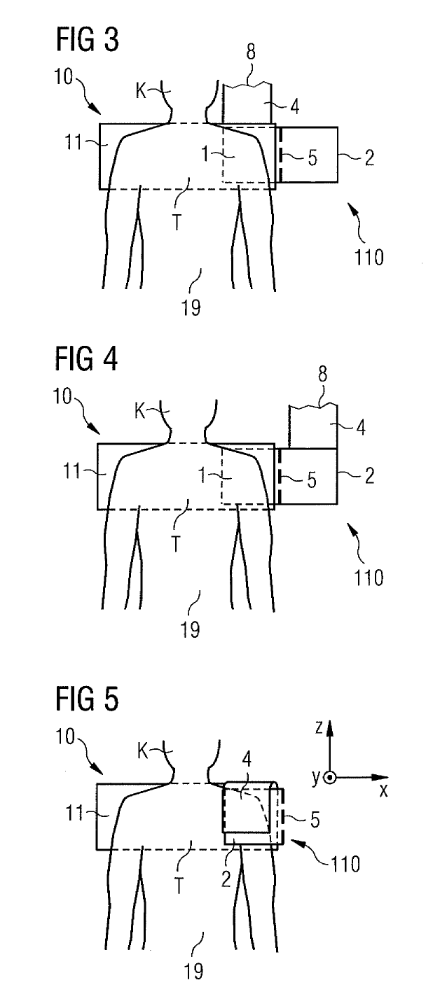

FIG. 3 shows a schematic top view of one embodiment of an arrangement of a shoulder local coil system 10 in preparatory position on a patient. The shoulder local coil system 10 is part of a high-frequency receiving device for generating magnetic resonance images of the shoulder region with corresponding evaluation electronics (not shown) and a dedicated local coil 110.

In FIG. 3, the local coil 110 is arranged in a region of the left shoulder of the patient in preparatory condition. As a mirror image, this also applies for the arrangement on the right shoulder side.

The local coil 110 includes a first coil section 1 with first antenna elements (not shown), a second coil section 2 with second antenna elements (not shown) and a fourth coil section 4 with fourth antenna elements (not shown). FIG. 3 also shows a center marking 5 that identifies a center between the first coil section 1 and the second coil section 2.

As FIG. 3 shows, the first coil section 1 is arranged below a cover plate 11 in a region under the left shoulder of the patient 19. The local coil 110 is pulled so far out of alignment with the cover plate 11 arranged in the shoulder and thorax region T that a part of the first coil section 1 and the whole coil section 2 are arranged open in a extended arrangement (e.g., aligned in a plane) in the x-direction to the shoulder. The entire local coil is pulled out so far under the cover plate 11 that the center marking 5 lies at half the height of the shoulder when the local coil 110 is positioned on the shoulder of the patient 19 (see FIG. 5).

The fourth shoulder coil section 4 extends toward the head K of the patient and is provided in the same plane as the first and second coil sections 1, 2 in the z-direction of the shoulder (cranial). This fourth coil section 4 is embodied in one piece with the first coil section 1. According to this embodiment, the fourth coil section 4 is embodied as a flexible section with internal antenna elements (not shown) in order, in a cranial region of the shoulder, to be tilted around this or positioned on the patient 19. Either the whole or the upper section of the fourth coil section 4 is provided with a Velcro hook or loop region, which interacts with a corresponding Velcro hook or loop region of the rear side of the second coil section 2 (see FIG. 5).

FIG. 4 shows a schematic top view of one embodiment of the arrangement of a shoulder local coil system 10 in preparatory position on a patient 19.

As in the system shown in FIG. 3, the shoulder local coil system 10 is part of a high-frequency receiving device for generating magnetic resonance images of the shoulder region with the corresponding evaluation electronics (not shown) and the dedicated local coil 110.

In FIG. 4, the local coil 110 is arranged in the region of the left shoulder of the patient in preparatory condition. The local coil 110 includes a first coil section 1 with first antenna elements (not shown), a second coil section 2 with second antenna elements (not shown) and a fourth coil section 4 with fourth antenna elements (not shown). FIG. 2 shows the function of the center marking 5, which identifies the center between the first coil element 1 and the second coil element 2 and is used for the alignment of the local coil 110 when the local coil 110 is positioned on the shoulder.

The local coil 110 also includes a fourth coil section 4 that is attached in one piece on the second coil section 2 in the z-direction (e.g., in direction of the head K). The fourth coil section 4 with antenna elements (not shown) is embodied flexibly and moveably relative to the second coil element 2 so that, following the positioning of the second coil element 2, the fourth coil section 4 is arranged around the shoulder in the cranial region of the shoulder joint. In the preparatory condition shown in FIG. 4, all three coil sections 1, 2, 4 are arranged in a plane (e.g., the three coil sections 1, 2, 4 are shown in the tilted-out condition). Similarly to the embodiment shown in FIG. 3, this results in an L-shape of the local coil 110.

In the whole or upper section, the fourth coil section 4 includes a Velcro hook or loop region 8 that, after the positioning of the second and fourth coil sections 2, 4 on the shoulder, interacts with a corresponding Velcro hook or loop region of the rear side of the first coil section 1 (see FIG. 5). When in position, the fourth coil section 4 and the first coil section 1 overlap under the shoulder region, thus enabling fixed connection of the two coil sections 1, 4 by the Velcro fastener principle.

FIG. 5 shows a schematic top view of one embodiment of the arrangement of a shoulder local coil system 10 in operational position on a patient 19.

As shown in FIG. 5, in operational status, both the second coil section 2 and the fourth coil section 4 are tilted about the shoulder joint so that the center marking 5 lies at half the height of the shoulder. In addition, the second coil section 2 and the fourth coil section 4 overlap above the shoulder joint. The connection of these two coil sections is facilitated with the aid of the Velcro fastener principle. This results in the wrapping of four sides of the shoulder joint. The SNR is improved by the good covering with antenna elements compared to conventional shoulder coils.

The shoulder local coil system 10 is also advantageous with respect to the positioning of the patient 19 in the local coil 110 since the patient 19 does not have to be moved. Instead the local coil 110 is positioned around the shoulder region on the patient 19. This results in both improved comfort of use for the patient 19 and improved workflow for the user. Since there is no need for the patient 19 to change position, expensive measuring time may be saved (e.g., if both shoulders are to be measured). This also results in increased efficiency.

Because the coil sections (e.g., the second coil section 2 and the fourth coil section 4) are made of a flexible material, the antenna elements provided in these coil sections 2, 4 lie as closely as possible to the shoulder joint, even in the case of different shoulder sizes. The displaceability of the local coil 110 in the x-direction and the flexibility in the y-direction enable the coil sections 2, 4 to be adapted to the widest variety of body anatomies, large or small or wide or narrow shoulders. This is achieved by enlarging or reducing the two coil sections 2, 4 and the displaceable mounting of the local coil 110 under the cover plate 11.

To prevent coupling of the antenna elements in the second coil section 2 and the fourth coil section 4 in the overlap region, the antenna elements in these two coil sections 2, 4 may be switched off individually.

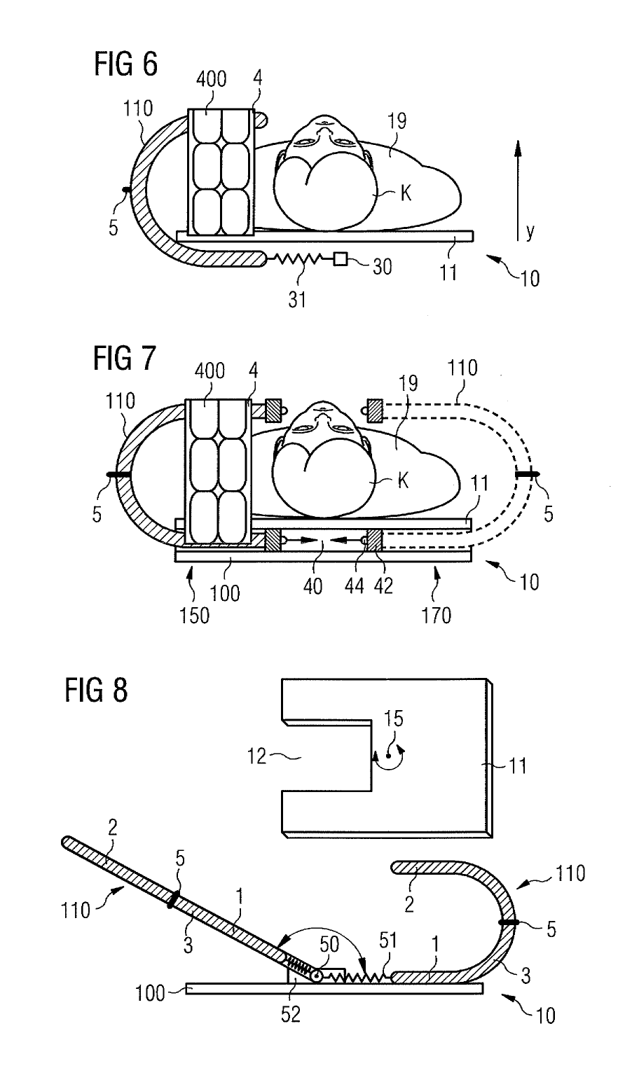

FIG. 6 shows a head-side front view of one embodiment of the arrangement of a shoulder local coil system 10 in operational position on the left shoulder of a patient 19.

The shoulder local coil system 10 includes a cover plate 11 and a local coil 110 that may be mounted jointly in a removable or rotatable manner on a base plate (not shown). In FIG. 6, the local coil 110 is arranged in a position on the left shoulder of the patient 19.

The local coil 110 is made of a flexible material and is guided laterally around the shoulder of the patient 19. The local coil includes the integrated first and second coil sections 1, 2 with the first and second antenna elements (not shown). The flexible local coil 110 is arranged below and above the shoulder such that the center marking 5 is at half the shoulder height. For this setting, the part of the local coil 110 arranged under the cover plate 11 may be laterally displaced.

For the lateral displacement of the local coil 110, the local coil 110 is attached by a return spring 31 to a fastening element 30 in the center of the cover plate. This enables, by pulling or inserting the local coil 110, the position of the local coil 110 to be adapted in the x-direction, while the patient 19 is lying on the cover plate 11. The flexibility of the local coil 110 (e.g., due to the material selected) also enables the size be adapted to the shoulder anatomy in the y-direction. The flexible local coil 110 may also be extended in a level position in order to make it easier for the patient 19 to lie down on the cover plate 11. This improves the workflow, and improved adaptation to the patient anatomy also enables the SNR to be improved compared to conventional shoulder coils.

The local coil 110 also includes a flexible fourth coil section 4 in the cranial shoulder region. This fourth coil section 4 includes a plurality of fourth antennas 400 arranged adjacent to each other that are substantially aligned in the x-y-plane. The plurality of fourth antennas 400 are shown schematically as circular antenna elements. The plurality of fourth antennas 400 may also be embodied as butterfly antennas since the plurality of fourth antennas 400 may only pick up little B₁ field. This fourth coil section 4 with the fourth antennas 400 achieves an additional SNR gain.

The local coil 110 shown in the third exemplary embodiment is directly connected to the lower side of the cover plate 11 via the return spring 31 and the fastening element 30 and, by turning the cover plate 11 by 180° in the x-z-plane, may be brought from the left shoulder position into the right shoulder position. To do this, the fourth coil section 4 is released, and the local coil 110 is brought into an extended position. After this, raising the patient 19 from the cover plate 11 or lowering the cover plate 11 while simultaneously supporting the head of the patient 19 enables the cover plate 11 to be rotated. After rotation by 180°, the local coil 110 reaches the position for the scanning on the right shoulder. Alternatively, the patient 19 may be positioned and the cover plate 11 changed by raising, rotating and corresponding positioning on the base plate 100 for the scan of the right shoulder. A simple change from the left to the right shoulder is thus possible. This achieves a time saving during the MRI scanning compared to conventional systems and a greater level of comfort for the patient 19.

FIG. 7 shows a head-side front view of one embodiment of the arrangement of a shoulder local coil system 10 in the operational position on the left shoulder of a patient 19.

The cover plate 11, the local coil 110 and the fourth coil section 4 are embodied similarly to the third exemplary embodiment. However, the local coil 110 includes, at each end of the local coil 110, which is rectangular and shown in bent condition, a slide element 42 that is guided in a slide rail 40 between the base plate 100 and the cover plate 11. Each slide element 42 has an eye 44, on which the slide element 42 may be pushed or pulled together with the local coil 110 through the slide rail 40. In extended condition (not shown in FIG. 7) such as, for example, shown in FIG. 3, the local coil 110 may be pulled from the position of the left shoulder (first side 150) to the position of the right shoulder (second side 170). Slide element 42, which was previously guided in the slide rail 40, is pulled out of guide bar, and the slide element 42 arranged on the opposite side of the local coil is guided in the slide rail 40 to the other side of the base plate 100. Following this, the local coil 110 may be placed around the opposite shoulder and without the position of the patient 19 on the cover plate 11 having to be changed. This saves time and is more comfortable for the patient 19.

FIG. 8 shows an outline front view of one embodiment of the arrangement of a shoulder local coil system 10 with a tiltable local coil element 110. This is shown in both the operational position (right side) and in a tilted position (left side) in the drawing.

Additionally, FIG. 8 shows a top view of one embodiment of the cover plate 11. The cover plate 11 includes a cutout 12, in which the first coil section 1 of the local coil element 110 guided thereunder in operational position comes to lie. The cover plate 11 has a rotary axis 15 to enable the cover plate 11 to be rotated about 180° in the x-z-plane so that the cutout 12 may be rotated from the left shoulder side toward the right shoulder side or in the reverse direction. Alternatively, rotation of up to 360° may be enabled.

In this embodiment, the local coil element 110 includes a first coil section 1, a second coil section 2 and a third coil section 3 arranged therebetween. Each of the first coil section 1, the second coil section 2, and the third coil section 3 includes integrated antenna elements (not shown). A center marking 5 is provided approximately in the center of the local coil elements 110. This marking is used in operational status to adjust the local coil position to half the shoulder height of the patient 19. In tilted condition, the first, second and third coil sections 1, 2, 3 are arranged linearly (e.g., in a plane). A rotary axis 50 is arranged in the z-direction at the outer end of the first coil section 1 about which the local coil 110 may be tilted from the left to the right side of the base plate 100. The local coil 110 may thus be used for both the scan of the left shoulder and the scan of the right shoulder.

In operating mode, following the positioning of the tiltable local coil 110 to either the left or right base-plate side, the cover plate 11 with the cutout 12 is positioned over the first coil section 1. This may lie on support elements 52 that, for example, support the rotary axis 50 for the local coil 110. The local coil 110 may thus be pulled out of the tunnel or shaft formed thereby in order to position the first coil section 1 under the corresponding shoulder position during the scan. The local coil 110 is embodied displaceably in the x-z-plane and is connected via a return spring 51 to the rotary axis 50 (e.g., with a mount into which the local coil may latch).

The local coil 110 is made of a flexible material (e.g., a plastic material), so that, in the third coil section 3, for example, the second coil section 2 of the local coil 110 may be bent upward on both the left side and the right side of the base plate 100 (e.g., for measuring the left or right shoulder). In operating condition, the local coil 110 adopts an approximately parallel position to the first coil section 1 so that a U-shaped local coil element 110 is formed. The local coil element 110, according to this exemplary embodiment, may thus be used universally for scanning both shoulders and provide a better SNR than conventional systems.

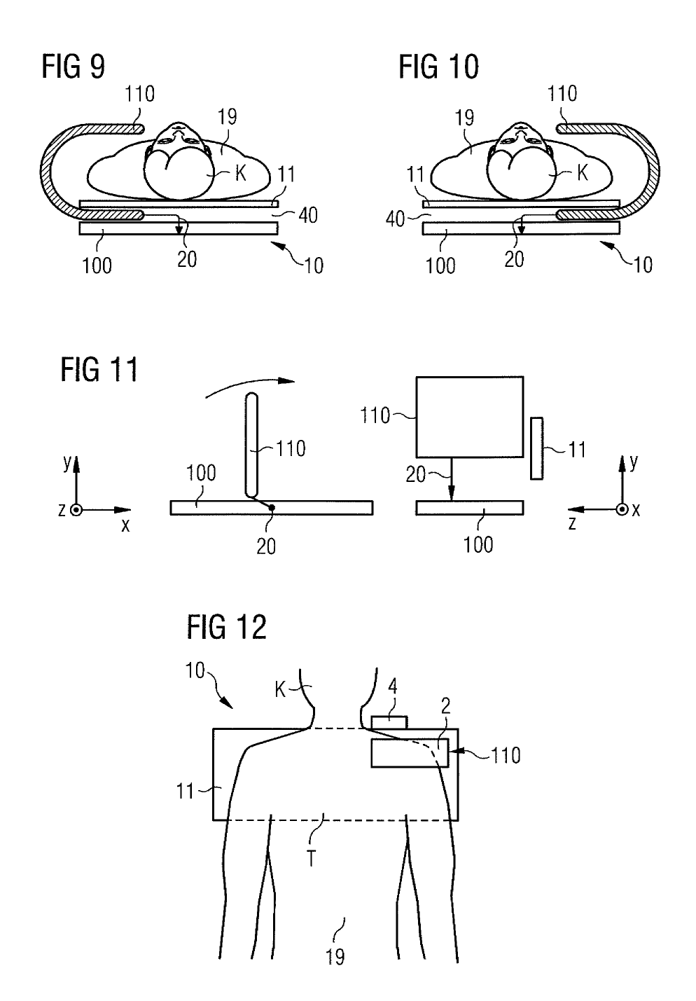

FIGS. 9 and 10 are head-side views of one embodiment of the arrangement of the shoulder local coil system 10 in FIG. 8 in operational position on the left shoulder and the right shoulder of a patient 19. FIGS. 9 and 10 show the slide rail 40 for the local coil element 110 formed from the base plate 100 and the cover plate 11. Both when scanning the left shoulder (FIG. 9) and when scanning the right shoulder (FIG. 10), this arrangement facilitates simple cable routing because the cable 20 is guided out of the slide rail 40 in the central position. The cable 20 connects the local coil 110 to the corresponding evaluation electronics of the high-frequency receiving device (not shown), for example.

FIG. 11 shows one embodiment of the arrangement of the shoulder local coil system 10 in FIG. 8 on the tilting of the local coil 110 from the left to the right shoulder position. On the left side of FIG. 11, this is shown in front view. On the right side of FIG. 11, the side view of the same tilt situation is depicted. According to this, in perpendicular tilted position, the rectangular flexible local coil 110 on the tilted-up cover plate 11 is tilted over to the other base-plate side. Following the complete tilting of the local coil 110 to the right side, the cover plate 11 may be tilted back into the original position.

FIG. 12 is a schematic top view of one embodiment of the arrangement of a shoulder local coil system 10 in operational position on a patient 19. This shows the local coil 110 positioned about the shoulder on the left shoulder side of the patient 19. This includes a simple rectangular shape including first, second and optional third coil sections 1, 2, 3, that permit posterior and anterior coverage of the shoulder joint with antenna elements.

In this exemplary embodiment, a fixed coil element 4 is arranged in the region above the shoulder (e.g., cranial to the shoulder as a fourth coil section). The fourth coil section 4 is embodied as fixed to or removable from the base plate 100. Otherwise, the function of this fourth coil section 4 is similar to the fourth coil sections that are used with the L-shaped coil elements, as shown in FIGS. 3 to 5. In this embodiment, butterfly-shaped antennas that capture the B₁ field may also be used.

The above-described shoulder local coil systems, the high-frequency receiving device, the magnetic resonance tomography system and method described in detail are only exemplary embodiments that may be modified by person skilled in the art in many different ways without leaving the scope of the invention. For example, further tilting or swinging mechanisms may also be used for changing sides. In addition, the use of the indefinite article “a” or “an” does not preclude the possibility that the features in question may also be present on a multiple basis. In addition, “units”, “apparatuses”, “means”, “elements” may include one or a plurality of components that may also be spatially distributed.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A shoulder local coil system for a magnetic resonance tomography system for generating magnetic resonance images of a shoulder region of person to be examined, the shoulder local coil system comprising:
a base plate;
a cover plate over the base plate, the cover plate being fixed or removable; and
a flexible local coil arranged wholly or partially movably between the base plate and the cover plate.

2. The shoulder local coil system as claimed in claim 1, wherein the flexible local coil comprises:
a first coil section comprising first antennas arranged in operation wholly or partially in alignment with the cover plate; and
a second coil section comprising second antennas arranged in operation outside a region between the base plate and the cover plate movably relative to the first coil section.

3. The shoulder local coil system as claimed in claim 1, wherein the first coil section is tiltable out of a first position, in which the first coil section and the second coil section lie in a first plane arranged substantially parallel to the base plate, into a second position, in which the second coil section lies wholly or partially in a second plane above the shoulder, the second plane being substantially parallel to the first plane.

4. The shoulder local coil system as claimed in claim 2, further comprising a flexible third coil section, the flexible third coil second comprising third antennas arranged between the first coil section and the second coil section.

5. The shoulder local coil system as claimed in claim 4, wherein the flexible third coil section has a center marking.

6. The shoulder local coil system as claimed in claim 2, wherein the first coil section, the second coil section, the third coil section, or a combination thereof has a basic structure comprising a plurality of parallel adjacent linking elements connected to each other in a moveable manner.

7. The shoulder local coil system as claimed in claim 2, wherein the cover plate has a cutout corresponding to a region of the first antennas.

8. The shoulder local coil system as claimed in claim 1, wherein the flexible local coil is arranged in a tunnel or shaft between the base plate and the cover plate such that the flexible local coil is partially or completely movable away from a first side of the base plate to an opposite second side of the base plate.

9. The shoulder local coil system as claimed in claim 1, wherein the flexible local coil is arranged on the base plate such that the flexible local coil is tiltable away from a first side of the base plate to an opposite second side of the base plate.

10. The shoulder local coil system as claimed in claim 4, further comprising a fourth coil section, the fourth coil section comprising fourth antennas.

11. The shoulder local coil system as claimed in claim 10, wherein the fourth coil section comprises a coil element connected to the base plate.

12. The shoulder local coil system as claimed in claim 10, wherein the fourth coil section comprises a flexible coil element that is arrangeable substantially perpendicular to the second coil section configured as a coil element that is tiltable around a cranial region of the shoulder.

13. A high-frequency receiving device for a magnetic resonance tomography system for generating magnetic resonance images of a shoulder region, the high-frequency receiving device comprising:
a shoulder local coil system comprising:
a base plate;
a cover plate over the base plate, the cover plate being fixed or removable; and
a flexible local coil arranged wholly or partially movably between the base plate and the cover plate.

14. The high-frequency receiving device as claimed in claim 13, wherein the flexible local coil comprises:
a first coil section comprising first antennas arranged in operation wholly or partially in alignment with the cover plate; and
a second coil section comprising second antennas arranged in operation outside a region between the base plate and the cover plate movably relative to the first coil section.

15. The high-frequency receiving device as claimed in claim 13, wherein the first coil section is tiltable out of a first position, in which the first coil section and the second coil section lie in a first plane arranged substantially parallel to the base plate, into a second position, in which the second coil section lies wholly or partially in a second plane above the shoulder, the second plane being substantially parallel to the first plane.

16. The high-frequency receiving device as claimed in claim 14, further comprising a flexible third coil section, the flexible third coil second comprising third antennas arranged between the first coil section and the second coil section.

17. A magnetic resonance tomography system for generating magnetic resonance images of a shoulder region, the magnetic resonance tomography system comprising:
a scanner with a basic field magnet; and
a high-frequency receiving device comprising:
a shoulder local coil system comprising:
a base plate;
a cover plate over the base plate, the cover plate being fixed or removable; and
a flexible local coil arranged wholly or partially movably between the base plate and the cover plate.

18. The magnetic resonance tomography system as claimed in claim 17, wherein the flexible local coil comprises:
a first coil section comprising first antennas arranged in operation wholly or partially in alignment with the cover plate; and
a second coil section comprising second antennas arranged in operation outside a region between the base plate and the cover plate movably relative to the first coil section.

19. The magnetic resonance tomography system as claimed in claim 18, further comprising a flexible third coil section, the flexible third coil second comprising third antennas arranged between the first coil section and the second coil section.

20. A method comprising:
generating magnetic resonance images of a shoulder region, the generating comprising using a shoulder local coil system, the should local coil system comprising a base plate, a cover plate over the base plate, the cover plate being fixed or removable, and a flexible local coil arranged wholly or partially movably between the base plate and the cover plate.
